(19) United States
(12) Patent Application Publication
(10) Pub. No.: US 2012/0031410 A1
(43) Pub. Date: Feb. 9, 2012

(54) Dental device for preventing a sleep disorder

(76) Inventor: Terry L. Jackson, St. Louis, MO (US)

(21) Appl. No.: 12/806,295

(22) Filed: Aug. 9, 2010

(51) Int. Cl. A61F 5/56

(52) U.S. Cl. 128/848

(57) ABSTRACT

A dental device for preventing a sleep disorder is disclosed which comprises a body having a lower teeth engaging surface, a central portion having a tongue engaging surface for holding a tongue down, and a pair of front ball clasps and a pair of back ball clasps for retaining the body to a lower jaw of an individual.

### BACKGROUND

This disclosure relates generally to a dental device used to prevent or inhibit a sleep disorder such as sleep apnea or snoring, and more particularly, to a dental device for preventing a sleep disorder by immobilizing or holding down a tongue to open an air passage or airway.

Sleep disorders such as sleep apnea or snoring are common conditions in adults. Sleep apnea is a sleep disorder characterized by pauses in breathing during sleep. An episode of sleep apnea can last long enough that one or more breaths are missed by an individual, and many episodes can occur throughout the night. Obstructive sleep apnea (OSA) is the most common category of sleep-disordered breathing. The muscles of the body typically relax during sleep and since the human throat or airway consists of a tongue and collapsible walls of soft tissue the airway can become obstructed when breathing during sleep. Chronic obstructive sleep apnea requires treatment to prevent low blood oxygen, sleep deprivation, and other complications. Obese individuals typically have low muscle tone and soft tissue around their airway and are at high risk for obstructive sleep apnea. Also, the elderly are more likely to have OSA than young people and men are more likely to have OSA than women. The risk of OSA increases with such features as high body weight, smoking, age, and diabetes. Common symptoms include snoring, restless sleep, and sleepiness during the daytime.

Snoring is the turbulent sound of air moving through the back of the mouth, nose, and throat. Many individuals who sleep next to a snorer have to suffer through the night listening to the annoying noise generated by a snoring individual. This situation impacts all involved in that all individuals may not obtain the required amount of sleep.

There are several devices available for the treatment of OSA and snoring. There are various oral or dental appliances that are worn inside of a mouth that are designed to move the lower jaw forward, or lift the soft palate of the mouth, or move the tongue forward in an effort to open up an airway. However, it does not appear that such oral or dental appliances have been acceptable.

Another device used to treat sleep apnea is a continuous positive airway pressure (CPAP) device. The CPAP device operates by keeping a patient's airway open during sleep by delivering a flow of pressurized air into the throat. The patient typically wears a plastic facial mask which is connected by a flexible tube to a small bedside CPAP machine which includes an air pump. The CPAP machine generates the required air pressure to keep the patient's airways open during sleep. Advanced CPAP models may warm or humidify the air and monitor the patient's breathing to insure proper treatment. Although CPAP therapy is effective, patients often find it extremely uncomfortable and individuals who sleep with the patient find the sound of the air pump to be annoying. After some use, many patients refuse to continue the therapy or fail to use the CPAP machines on a nightly basis.

There are also several surgical procedures that may be used to treat OSA and snoring by anatomically altering a patient's airway. Several levels of obstruction may be addressed, including altering the nasal passage, the throat (pharynx), the base of tongue, and the facial skeleton. Surgical treatment for obstructive sleep apnea may be individualized in order to address all anatomical areas of obstruction. Often, correction of the nasal passages needs to be performed in addition to correction of the oropharynx passage. Uvulopalatopharyngoplasty (UPPP) has been used to address pharyngeal obstruction. UPPP is a procedure in which tissue at the posterior portion of the soft palate is surgically removed. However, such surgical techniques have met with varying success and individuals may want to avoid surgery.

Therefore, there is a need to provide a device that may be employed to be able to prevent or reduce a sleep disorder such as snoring or OSA. It would also be advantageous to be able to provide a device that will immobilize the tongue during sleep to prevent the tongue from blocking the respiratory passage or airway.

### SUMMARY

In one form of the present disclosure, a dental device for preventing a sleep disorder is disclosed which comprises a body having a lower teeth engaging surface, a central portion having a tongue engaging surface for holding a tongue down, and a pair of front ball clasps and a pair of back ball clasps for retaining the body to a lower jaw of an individual.

In another form of the present disclosure, a dental device for preventing a sleep disorder comprises a body having a left side and a right side and each of the sides having a lower teeth engaging surface, a first center portion extending between the left side and the right side, a first pair of ball clasps extending from the left side and adapted to engage teeth, a second center portion extending between the left side and the right side, and a second pair of ball clasps extending from the right side and adapted to engage teeth.

In yet another form of the present disclosure, a dental device for preventing a sleep disorder comprises a left pad portion having a lower teeth engaging surface, a right pad portion having a lower teeth engaging surface, a body connected between the left pad portion and the right pad portion, the body having a tongue engaging surface, a first pair of ball clasps extending from the left pad portion and adapted to engage teeth, and a second pair of ball clasps extending from the right pad portion and adapted to engage teeth.

In light of the foregoing comments, it will be recognized that a principal object of the present disclosure is to provide a dental device for preventing a sleep disorder by holding a tongue down so that the tongue does not block an airway or air passage.

A further object of the present disclosure is to provide a dental device which is of simple construction and design, is inexpensive, and which can be easily employed with highly reliable results.

Another object of the present disclosure is to provide a dental device that is easy to use, comfortable to wear, and may be easily removed from a mouth.

A still further object of the present disclosure is to provide a dental device that can be stored when not in use.

A further object of the present disclosure is to provide a dental device that may be used to display the camphor container assembly.

Another object of the present disclosure is to provide a dental device that may be fitted to a lower jaw to immobilize a tongue so that the tongue does not block an airway or air passage.

Yet another object of the present disclosure is to provide a dental device that may be easily manufactured and custom made based upon a mold of a mouth of an individual.

These and other objects and advantages of the present disclosure will become apparent after considering the following detailed specification in conjunction with the accompanying drawings, wherein:

### BRIEF DESCRIPTION OF THE DRAWINGS

FIG. 1 is a top perspective view of an embodiment of a dental device for preventing a sleep disorder constructed according to the present disclosure;

FIG. 2 is a bottom perspective view of the the dental device for preventing a sleep disorder constructed according to the present disclosure;

FIG. 3 is a left view of the dental device for preventing a sleep disorder;

FIG. 4 is a partial front perspective view of a mouth of an individual having the dental device for preventing a sleep disorder inserted into the mouth;

FIG. 5 is bottom perspective view of another embodiment of a dental device for preventing a sleep disorder constructed according to the present disclosure; and

FIG. 6 is a top perspective view of the dental device for preventing a sleep disorder constructed according to the present disclosure.

### DETAILED DESCRIPTION OF THE EMBODIMENTS

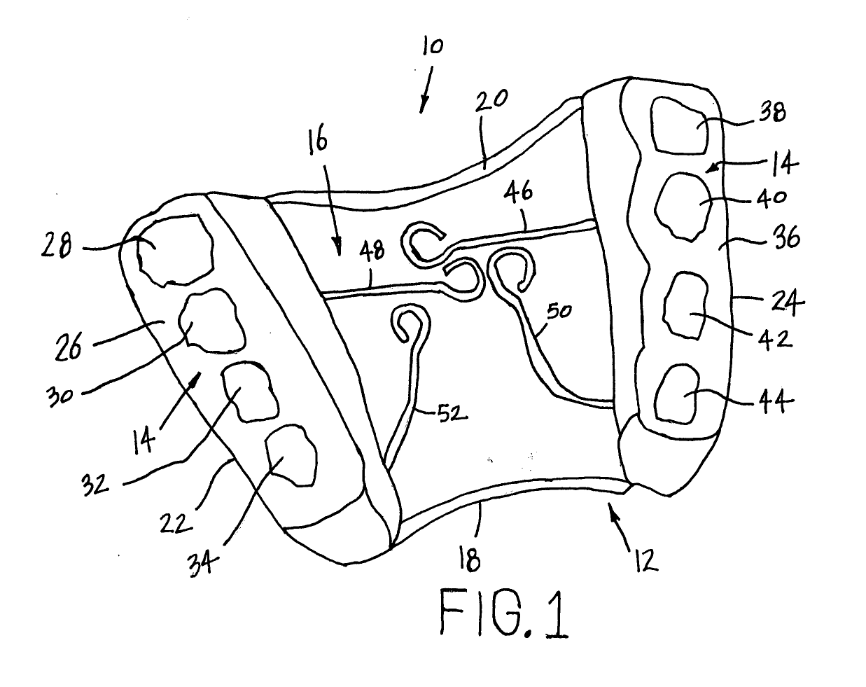

Referring now to the drawings, wherein like numbers refer to like items, number 10 identifies an embodiment of a dental device constructed according to the present disclosure. With reference now to FIG. 1, the dental device 10 comprises a body 12 having an upper teeth engaging surface 14 and a central portion 16. The body 12 has a front 18, a back 20, a left side 22, and a right side 24. The dental device 10 may be formed from any appropriate natural or synthetic material or materials, such as synthetic or natural rubber, medical rubber, plastics such as polyvinyl plastics material including vinyl acetate ethylene polymeric material, polyethylene vinyl acetate or a polyethylene or polypropylene. The material may be clear or colored. The dental device 10 may be custom made by taking a casting or a dental impression of an individual's mouth, tongue, and teeth. From the casting the dental device 10 is produced which is adapted to fit on the lower teeth and have the upper teeth mate with or against the upper teeth engaging surface 14. The upper teeth engaging surface 14 has a left side pad portion 26 in which a first recess 28, a second recess 30, a third recess 32, and a fourth recess 34 are formed therein. The first recess 28 is sized and shaped to receive an upper molar, the second recess 30 is adapted to receive an upper molar, the third recess 32 is adapted to receive an upper premolar, and the fourth recess 34 is adapted to receive an upper canine tooth. The upper teeth engaging surface 14 also has a right side pad portion 36 in which a first recess 38, a second recess 40, a third recess 42, and a fourth recess 44 are formed therein. The recesses 38, 40, 42, and 44 are sized and shaped to receive or mate with upper teeth such as molars, premolars, and a canine tooth. It is possible that the recesses 28, 30, 32, 34, 38, 40, 42, and 44 may be formed to mate with other teeth and also more or less recesses may be formed in the surface 14 depending upon a particular application.

Embedded in the body 12 are four pins 46, 48, 50, and 52 which have ball clasps ends which are not shown in this particular view, but will be discussed in more detail herein. The pins 46, 48, 50, and 52 are made of metal such as stainless steel. Further, the front 18 has a width and the back 20 has a width with the back 20 being wider than the front 18. The width of the back 20 being greater than the front 18 is due to a mouth being wider in the back than in the front. Also, the span of the teeth in the front of the mouth is smaller than in the back of the mouth.

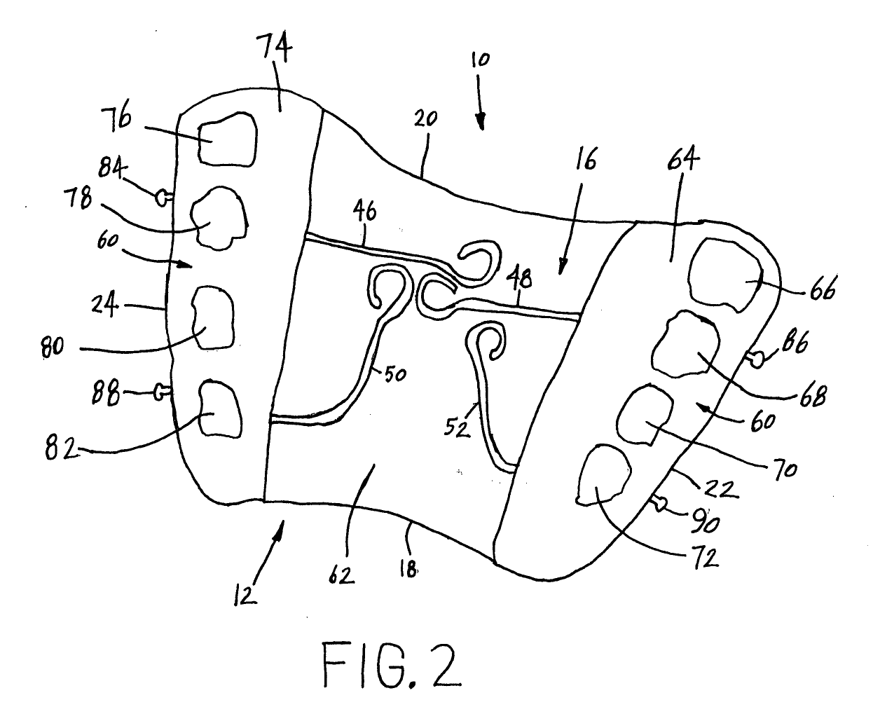

Referring now to FIG. 2, a bottom perspective view of the dental device 10 is shown. The dental device 10 comprises the body 12 having a lower teeth engaging surface 60 and the central portion 16 having a tongue engaging surface 62 for holding a tongue down during use. The body 12 is also shown having the front 18, the back 20, the left side 22, and the right side 24. The lower teeth engaging surface 60 has a left side pad portion 64 in which a first recess 66, a second recess 68, a third recess 70, and a fourth recess 72 are formed therein. The first recess 66 is sized and shaped to receive a lower molar, the second recess 68 is adapted to receive a lower molar, the third recess 70 is adapted to receive a lower premolar, and the fourth recess 72 is adapted to receive a lower canine tooth. The lower teeth engaging surface 60 also has a right side pad portion 74 in which a first recess 76, a second recess 78, a third recess 80, and a fourth recess 82 are formed therein. The first recess 76 is sized and shaped to receive a lower molar, the second recess 78 is adapted to receive a lower molar, the third recess 80 is adapted to receive a lower premolar, and the fourth recess 82 is adapted to receive a lower canine tooth.

The pin 46 has a ball clasp 84 extending out from the right side 24 of the dental device 10. The ball clasp 84 is used to retain the dental device 10 securely in place during use. The ball clasp 84 may be positioned to fit between two teeth such as two molars. The pin 48 has a ball clasp 86 extending out from the left side 22 of the dental device 10. The ball clasp 86 may be orientated to fit between two teeth such as two molars. The ball clasps 84 and 86 form a pair of back ball clasps for retaining the body 12 to a lower jaw (not shown) of an individual during use of the dental device 10. A ball clasp 88, which is part of the pin 50, extends from the right side 24 of the dental device 10. The pin 52 is shown to have a ball clasp 90 extending out from the left side 22 of the dental device 10. The ball clasps 88 and 90 are an example of a pair of front ball clasps for retaining the body 12 to a lower jaw of an individual. The ball clasps 88 and 90 may be positioned between a lower premolar and a lower canine tooth.

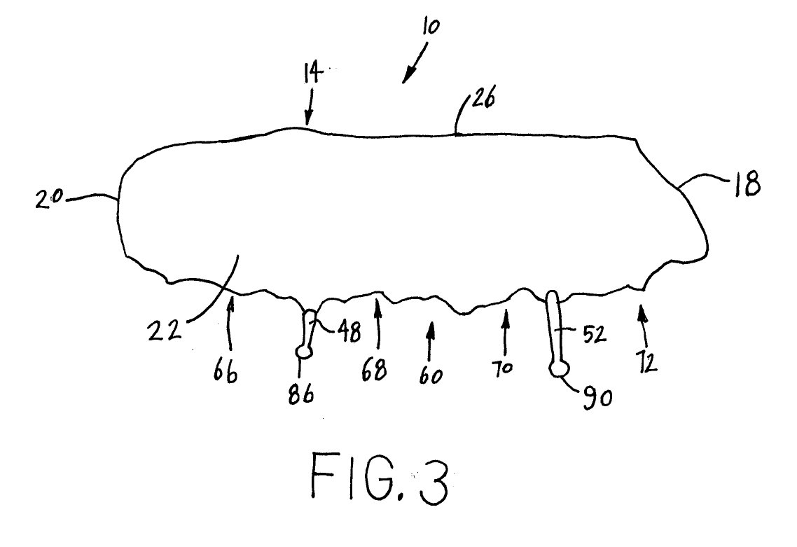

A left side view of the dental device is depicted in FIG. 3. The dental device 10 is shown having the left side 22, the front 18, and the back 20. The recesses 66, 68, 70, and 72 are also illustrated and show how the dental device 10 is constructed to fit against or mate with corresponding teeth such as molar, premolars, and canine teeth of a lower jaw of an individual. The ball clasp 86, which is part of the pin 48, is shown extending out and down from the dental device 10. The ball clasp 90, also part of the pin 52, extends out and down from the dental device 10. Depending upon the particular contours and size of an individual's mouth, tongue, and teeth, the pin 52 may extend further down or be longer than the pin 48. As has been explained, the contours are developed from taking a casting of an individual's mouth. Although not shown in detail in this figure, the left side pad portion 26 has the recesses 28, 30, 32, and 34 that are formed to mate with teeth of the upper jaw.

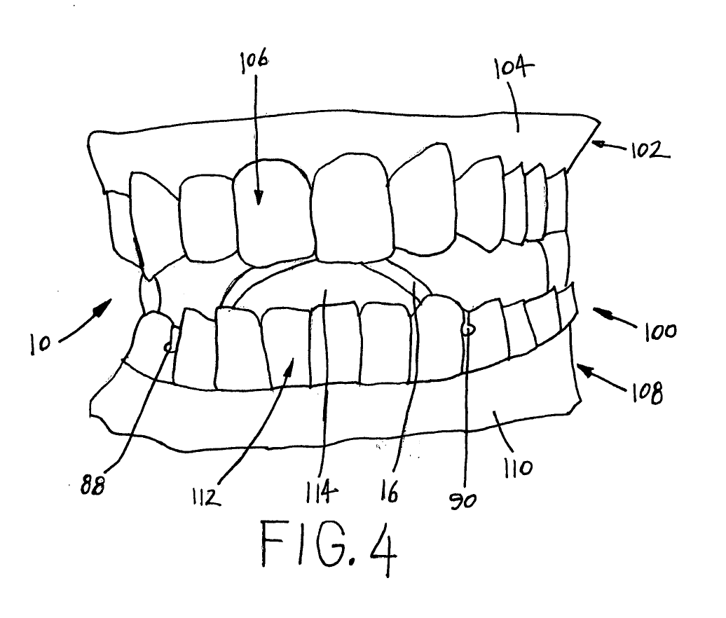

FIG. 4 shows a front view of the dental device 10 positioned within a mouth 100. The mouth 100 has an upper jaw 102 having gums 104 and upper teeth 106 and a lower jaw 108 having gums 110 and lower teeth 112. The dental device 10 is secured to the lower teeth 112 by use of the ball clasps 88 and 90 and also ball clasps 84 and 86 which are hidden in this view. The upper teeth 106 are adapted to be positioned on the dental device 10. Also, the dental device 10 moves the lower jaw 108 forward to open the airway. The dental device 10 has the central portion 16 which is arched upwardly to accommodate and hold or immobilize a tongue 114. The tongue 114 is held in place by the central portion 16 and the tongue engaging surface 62 (not shown). By immobilizing or holding the tongue 114 in place during use, the dental device 10 prevents the tongue 114 from moving backwards to restrict or obstruct the airway or air passage. By preventing movement of the tongue 114, the dental device 10 prevents snoring or sleep apnea. Although the central portion 16 is shown as being arched upwardly it is also possible that the central portion 16 may be arched downwardly or be straight depending upon the particular contours and shape of an individual's tongue.

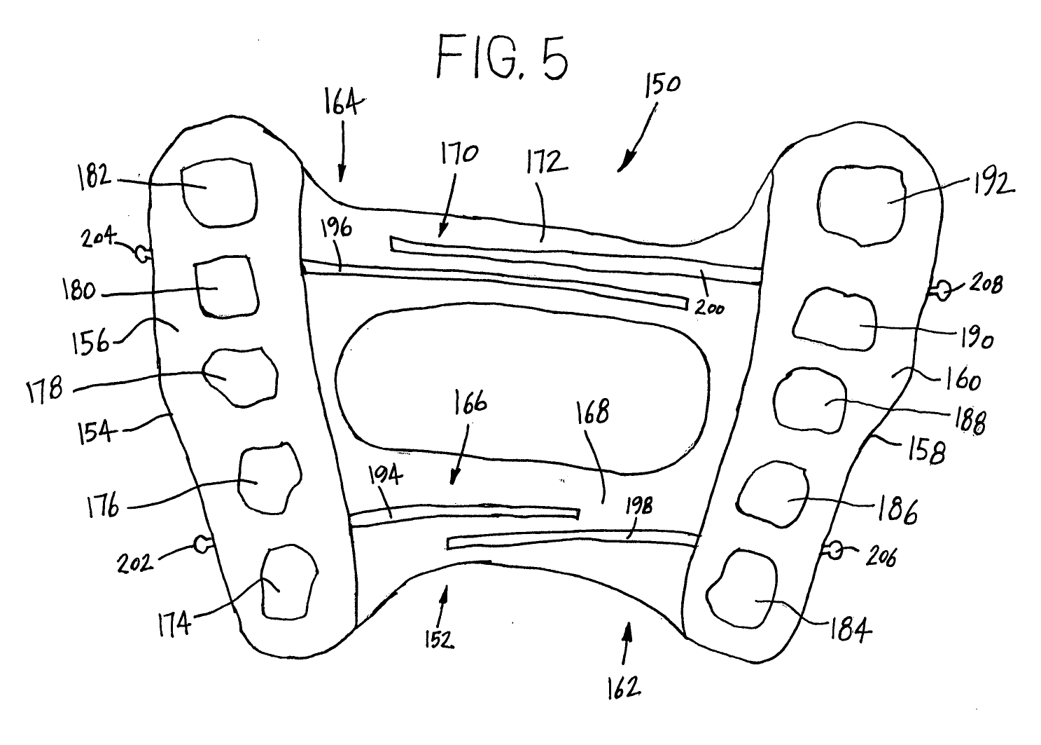

With reference now to FIG. 5, another embodiment of a dental device for preventing a sleep disorder 150 is illustrated. The dental device 150 comprises a body 152 having a left side 154 having a left lower teeth engaging surface 156, and a right side 158 having a right lower teeth engaging surface 160. The dental device 150 has a front 162 and a back 164. A first center portion 166 extends between the left side 154 and the right side 158. The first center portion 166 has a tongue engaging surface 168. The dental device 150 also has a second center portion 170 that extends between the left side 154 and the right side 158. The second center portion 170 has a tongue engaging surface 172.

The left lower teeth engaging surface 156 has formed therein a first recess 174, a second recess 176, a third recess 178, a fourth recess 180, and a fifth recess 182. The recesses 174, 176, 178, 180, and 182 are adapted to mate with lower teeth. The right lower teeth engaging surface 160 also has formed therein a first recess 184, a second recess 186, a third recess 188, a fourth recess 190, and a fifth recess 192. All of the recesses 184, 186, 188, 190, and 192 are sized and shaped to mate or accept therein lower teeth. It is possible that more or less recesses may be formed in the dental device 150.

Embedded within the body 152 are four pins 194, 196, 198, and 200 which have ball clasps 202, 204, 206, and 208, respectively. The ball clasps 202, 204, 206, and 208 are used to secure the dental device 150 to the lower teeth of an individual. The pins 194, 196, 198, and 200 and the ball clasps 202, 204, 206, and 208 are formed from metal such as stainless steel. The ball clasps 202 and 204 are an example of a first pair of ball clasps extending from the left side 154 which are adapted to engage teeth and the ball clasps 206 and 208 are an example of a second pair of ball clasps extending from the right side 158 which are adapted to engage teeth.

The first center portion 166 has a width and the second center portion 170 has a width with the width of the second center portion 170 being wider than the first center portion 166. This allows for the dental device 150 to fit within a mouth in a comfortable manner. The first center portion 166 and the second center portion 170 may be arched upwardly to allow a tongue to fit underneath the center portions 166 and 170 and to contact the tongue engaging surfaces 168 and 172. By use of the center portions 166 and 170 and the tongue engaging surfaces 168 and 172, a tongue is held in place or immobilize during sleep. The dental device 150 prevents the tongue from moving back to obstruct the airway or air passage during sleep. Although the central portions 166 and 170 are shown being arched upwardly, it is also possible to have the central portions 166 and 170 arched downwardly or straight. The particular design being dependent upon the shape and size of the tongue and teeth of a particular individual.

As can be appreciated, the dental device 150 may be formed from any appropriate natural or synthetic material or materials, such as synthetic or natural rubber, medical rubber, plastics such as polyvinyl plastics material including vinyl acetate ethylene polymeric material, polyethylene vinyl acetate or a polyethylene or polypropylene. The material may be clear or colored. The dental device 150 may be custom made by taking a casting or a dental impression of an individual's mouth, tongue, and upper and lower teeth. From the casting the dental device 150 is produced which is adapted to fit on the lower teeth with the central portions 166 and 170 for holding the tongue in place.

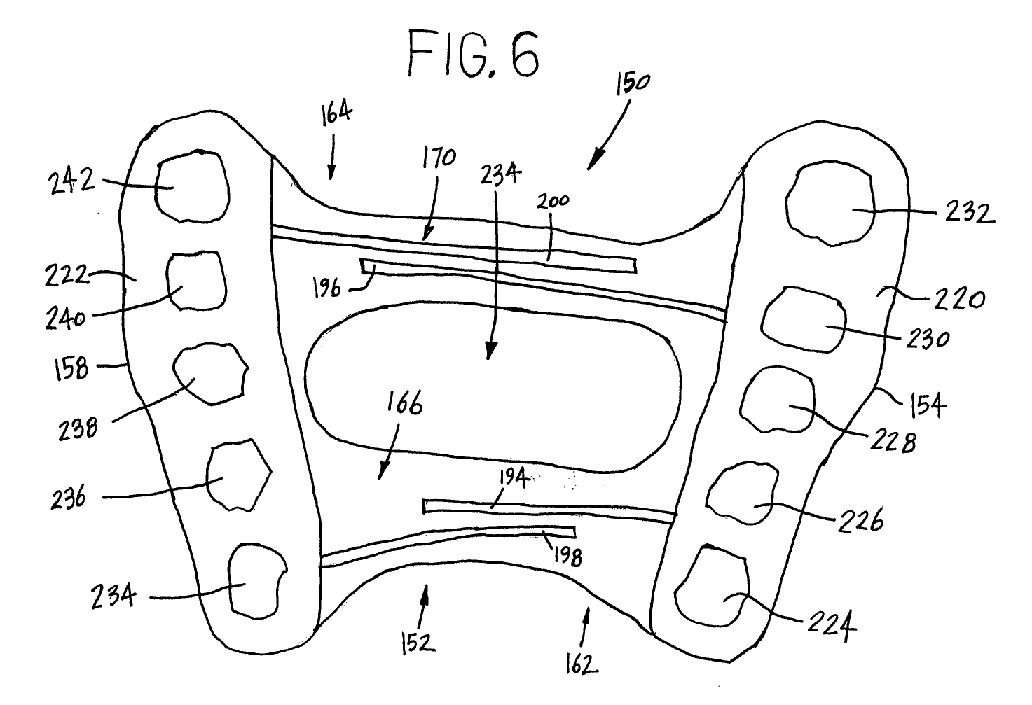

FIG. 6 shows a top perspective view of the dental device 150. The dental device 150 comprises the body 152 having the left side 154 having a left upper teeth engaging surface 220, and a right side 158 having a right upper teeth engaging surface 222. The dental device 150 has the front 162 and the back 164. The first center portion 166 extends between the left side 154 and the right side 158. The second center portion 170 also extends between the left side 154 and the right side 158. The left upper teeth engaging surface 220 has a first recess 224, a second recess 226, a third recess 228, a fourth recess 230, and a fifth recess 232 for engaging or mating with upper teeth. The right upper teeth engaging surface 222 has a first recess 234, a second recess 236, a third recess 238, a fourth recess 240, and a fifth recess 242 formed therein for receiving or mating with upper teeth. It is possible that more or less recesses may be formed in the dental device 150 depending upon the particular application. Further, in the pins 194, 196, 198, and 200 are shown, however, the ball clasps 202, 204, 206, and 208 are hidden by the surfaces 220 and 222.

The center portions 166 and 170 form a cutout region or opening 234 through which the a tongue may be positioned. The opening 234 allows for air to flow past the tongue when the dental device 150 is placed in the mouth. The opening 234 also allows for any fluids that may accumulate under the dental device 150 to be drawn away from the dental device 150.

As can be appreciated, the dental devices 10 and 150 may take on any desired shape dependent upon the particular mouth in which the devices 10 and 150 are to be inserted and used. It should be recognized that the dental devices 10 and 150 are formed of relatively lightweight material so that the dental devices 10 and 150 can be quickly and easily manufactured, assembled, transported, and stored. Further, the dental devices 10 and 150 can be constructed of relatively inexpensive materials that will provide for the dental devices 10 and 150 to be mass produced, disposable, and suitable for long time use.

From all that has been said, it will be clear that there has thus been shown and described herein a dental device for preventing a sleep disorder which fulfills the various objects and advantages sought therefor. It will become apparent to those skilled in the art, however, that many changes, modifications, variations, and other uses and applications of the subject dental device for preventing a sleep disorder are possible and contemplated. All changes, modifications, variations, and other uses and applications which do not depart from the spirit and scope of the present disclosure are deemed to be covered by the present disclosure, which is limited only by the claims which follow.

What is claimed is:

1. A dental device for preventing a sleep disorder comprising:
a body having a lower teeth engaging surface;
a central portion having a tongue engaging surface for holding a tongue down; and
a pair of front ball clasps and a pair of back ball clasps for retaining the body to a lower jaw of an individual.

2. The dental device of claim 1 wherein the pair of back ball clasps are each positioned between a pair of molars.

3. The dental device of claim 1 wherein the pair of front ball clasps are each positioned between a premolar and a canine tooth.

4. The dental device of claim 1 wherein the lower teeth engaging surface comprises recesses adapted to mate against each lower tooth.

5. The dental device of claim 1 wherein the body further comprises an upper teeth engaging surface.

6. The dental device of claim 5 wherein the upper teeth engaging surface comprises recesses adapted to mate against each upper tooth.

7. The dental device of claim 1 wherein the body further comprises a front having a width and a back having a width and the width of the back is wider than the width of the front.

8. The dental device of claim 1 wherein the central portion is arched upwardly to fit a tongue therein.

9. The dental device of claim 1 wherein the body covers the lower teeth.

10. A dental device for preventing a sleep disorder comprising:
a body having a left side and a right side and each of the sides having a lower teeth engaging surface;
a first center portion extending between the left side and the right side;
a first pair of ball clasps extending from the left side and adapted to engage teeth;
a second center portion extending between the left side and the right side; and
a second pair of ball clasps extending from the right side and adapted to engage teeth.

11. The dental device of claim 10 wherein each of the lower teeth engaging surfaces comprises recesses adapted to mate with lower teeth.

12. The dental device of claim 10 wherein each of the sides further comprises an upper teeth engaging surface.

13. The dental device of claim 12 wherein each of the upper teeth engaging surfaces comprises recesses adapted to mate with upper teeth.

14. The dental device of claim 10 wherein the first central portion and the second central portion is arched upwardly to contain a tongue therein.

15. The dental device of claim 10 wherein the first central portion has a width and the second central portion has a width and the width of the second central portion is greater than the width of the first central portion.

16. A dental device for preventing a sleep disorder comprising:
a left pad portion having a lower teeth engaging surface;
a right pad portion having a lower teeth engaging surface;
a body connected between the left pad portion and the right pad portion, the body having a tongue engaging surface;
a first pair of ball clasps extending from the left pad portion and adapted to engage teeth; and
a second pair of ball clasps extending from the right pad portion and adapted to engage teeth.

17. The dental device of claim 16 wherein the left pad portion further comprises an upper teeth engaging surface and the right pad portion further comprises an upper teeth engaging surface.

18. The dental device of claim 16 wherein the body is arched upwardly to contain a tongue therein.

19. The dental device of claim 16 wherein the body has a front having a width and a back having a width and the width of the back is greater than the width of the front.

20. The dental device of claim 16 wherein the lower teeth engaging surface of the left pad comprises recesses adapted to mate with lower teeth and the lower teeth engaging surface of the right pad comprises recesses adapted to mate with lower teeth.
